(19) United States
(12) Patent Application Publication
(10) Pub. No.: US 2011/0130837 A1
(43) Pub. Date: Jun. 2, 2011

(54) Intervertebral Implant

(75) Inventor: Helmut D. Link, Hamburg (DE)

(73) Assignee: CERVITECH, INC., San Diego, CA (US)

(21) Appl. No.: 13/056,079

(22) PCT Filed: Jul. 29, 2009
(86) PCT No.: PCT/EP09/05492

§ 371 Date: Jan. 26, 2011

(30) Foreign Application Priority Data
Jul. 29, 2008 (EP) 08013613.8

(51) Int. Cl. A61F 2/44

(52) U.S. Cl. 623/17.11

(57) ABSTRACT

An intervertebral implant for insertion into an intervertebral disc space having a contact face for connection to an adjacent vertebral body. The contact face has a porous coating that allows bone tissue ingrowth into the porous coating to form an intimate connection between the contact face and the bone tissue. Rounded teeth are provided that protrude above the contact face and the porous coating and have a radius of not less than 0.1 mm. The rounded teeth do not penetrate through the bone tissue but merely compact the bone tissue. The teeth provide fixation in the bone tissue even without intimate connection to the bone tissue. A method of securing the implant in the intervertebral space with easy removal in the event of a replacement is also disclosed.

### CROSS REFERENCE TO RELATED APPLICATIONS

This application is a national stage filing under 35 USC 371 of International Application No. PCT/EP2009/005492, filed Jul. 29, 2009, which claims Convention priority from European Application No. 08013613.8, filed Jul. 29, 2008, the full disclosure of which are incorporated herein by reference.

The invention relates to an intervertebral implant for insertion into an intervertebral space. The intervertebral implant comprises a contact face that is designed to bear on a surface of a vertebral body defining the intervertebral space. Several teeth are arranged on the contact face and protrude from the contact face. The end of the teeth remote from the contact face runs out in an edge.

### BACKGROUND OF THE INVENTION

Hitherto, intervertebral implants were in most cases connected to the vertebral body mechanically, for example by a flange of the implant being screwed onto the vertebral body. Implants are increasingly being designed, for example by application of a porous coating to the relevant surface areas, such that the bone tissue can form an intimate connection with the surface of the implant. The intimate connection provides the implant with hold, and it is possible to dispense with an additional mechanical connection.

An aim has hitherto been to ensure that the intimate connection between the bone tissue and the surface of the implant is as stable and as durable as possible. This aim is based on the assumption that the implant should remain in the body for life. There are then no other demands on the connection between the implant and the bone except that it has to be sufficiently strong to meet all conceivable load situations.

Implants are increasingly being used in younger patients whose life expectancy is greater than the expected life of the implant. It has to be taken into consideration from the outset that the implant may have to be removed from the bone again at a later time in order to replace it with a new one.

If the bone tissue has formed an intimate connection with the surface of the implant, the surgeon will attempt to drive a sharp tool between the bone and the implant in order to break the connection when removing the implant. This can be done without too much difficulty if the surface of the implant has a flat or only slightly curved shape. The connection is much more difficult to break if teeth protrude from such a surface and have penetrated into the bone substance and are connected to the bone tissue deep within the bone. This is because the surgeon cannot move instruments deep into the bone.

If the surface of the teeth is configured such that the bone tissue can form an intimate connection, the teeth readily contribute to a secure connection between the bone and the implant. However, if one wants to do without an intimate connection between the bone tissue and the surface of the teeth, so as to make it easier subsequently to remove the implant, it is then no longer certain that the teeth will provide a sufficient contribution to fixing the implant.

### SUMMARY OF THE INVENTION

Proceeding from the prior art mentioned at the outset, the object of the invention is to provide an intervertebral implant in which the teeth are designed such that they provide the implant with a good hold in the bone, even without intimate connection to the bone tissue. The object is achieved by the features of the invention as broadly described herein. According to the invention, the edge of the tooth remote from the contact face is rounded, and the radius of the edge is not less than 0.1 mm. Advantageous embodiments are set forth in the preferred embodiments.

A number of terms will first be explained. An edge forms where two surfaces meet. The edge has a longitudinal extent that is oriented parallel to each of the two surfaces. An edge at an end of the tooth remote from the contact face is at a distance from the contact face along its entire length. The edge is rounded transversely to its longitudinal extent.

The rounded teeth according to the invention penetrate with their entire volume into the bone tissue, such that the contact face from which the teeth protrude bears flat on the surface of the bone tissue. Since the edge of the teeth that first penetrates into the bone tissue is not sharp but rounded, the bone tissue is not cut through during the penetration of the teeth but merely compacted. The bone tissue compacted around the teeth is better able to take up compressive forces from the teeth than a bone tissue that has been weakened by the penetration of a sharp-edged tooth.

The teeth serve mainly to transmit to the bone tissue, by way of their flanks, compressive forces that act substantially parallel to the contact face. Tensile forces that act in a direction perpendicular to the contact face should mainly be transmitted from the contact face itself to the bone tissue. For this purpose, the contact face can be covered with a porous coating, such that the bone tissue can grow into the pores and in this way forms an intimate connection with the contact face. The pore width defined according to ASTM F1854 can be between 30 μm and 70 μm, and the pore coverage between 20% and 40%. Since no intimate connection is intended to form between the surface of the teeth and the bone tissue, the teeth preferably stand free from the coating. The surfaces of the teeth that adjoin the rounded edge are therefore not porous. Moreover, the surface of the teeth should not be too rough. The roughness Rₐ defined according to DIN EN ISO 4288 and 3274 is preferably less than 12 μm, more preferably less than 8 μm. An intervertebral implant in which the teeth stand free from the coating, and in which the uncoated tips of the teeth protrude from the coating, could constitute an independent invention even without the edges of the teeth being rounded.

The intervertebral implant according to the invention is preferably used in the cervical region of the spinal column. So as not to intervene too far in the vertebral body, the height of the teeth above the contact face should be less than 1.5 mm, preferably less than 1.2 mm. These figures relate to the body of the implant. If a coating is applied to the body of the implant, then the height of the teeth relative to the coating is reduced in accordance with the thickness of the coating. The height of the teeth should not be less than 0.8 mm. The radius of the rounded edge is then of the order of between 5% and 15% of the height of the teeth. The width of the teeth is preferably less than 3 mm, more preferably less than 2 mm.

When inserting an intervertebral implant according to the invention, the surgeon gains access to the spinal column from the ventral direction. Using a tool engaged on the ventral end of the intervertebral implant, the implant is inserted into the intervertebral space in the dorsal direction. The direction between the ventral end and dorsal end of the prosthesis is designated as the AP direction. The lateral direction is at right angles thereto.

To make the insertion of the implant into the intervertebral space easier, the surface of the teeth that is oriented in the dorsal direction can have a shallower pitch relative to the contact face than does the surface of the teeth that is oriented in the ventral direction. The pitch of this dorsal flank relative to the contact face can be between 40° and 60°, for example. Teeth of this kind are known from the prior art in which the ventral flank of the teeth, which lies opposite the dorsal flank and which counteracts a withdrawal of the implant in the ventral direction, is perpendicular to the contact face. Although a flank that is as perpendicular as possible is advantageous for good transmission of forces in the ventral direction, it has been found in the development of the invention that teeth with a flank oriented perpendicular to the contact face tend to cut through bone tissue when they penetrate into the bone. In the context of the invention, the danger of damaging the bone tissue can be reduced if the ventral flank is not perpendicular to the contact face but instead at an angle of between 78° and 88°, preferably of between 80° and 85°. The same applies to the lateral flanks of the teeth. Again, the teeth are able to penetrate more gently into the bone tissue if the lateral flanks are not perpendicular but instead at an angle of slightly less than 90° to the contact face. The preferred angle range is the same as for the ventral flank.

The contact face of the intervertebral implant can be flat when seen in the AP direction. The contact face can also be curved in the AP direction to correspond to the shape of an adjacent vertebral body.

If the teeth merely compact the bone tissue without forming an intimate connection with the bone tissue, the contact face becomes all the more important for the stable connection between the implant and the bone tissue. It is therefore desirable for the contact face to be as large as possible. Looking at the horizontal contour of the intervertebral implant according to the invention, this is preferably occupied entirely by the contact face, except for the teeth. At least 80%, preferably 90%, of the surface area remaining alongside the teeth should be occupied by the contact face. The teeth are preferably arranged such that the contiguous areas of the contact face between the teeth are as large as possible. For example, several teeth can be arranged in a row that extends substantially in the AP direction. The teeth are preferably so small that, in total, they occupy not more than 15%, preferably not more than 10%, more preferably not more than 8% of the surface area enclosed by the contour of the intervertebral implant. All these measures ensure that the surface area across which the bone tissue can form an intimate connection with the contact face is as great as possible. In this respect, an embodiment is particularly preferred in which the above-mentioned smallness of the teeth is combined with the different pitch of the ventral flank in relation to the dorsal flank, in which the teeth protrude from the contact face otherwise provided with the porous coating and stand free from the coating. Such a combination of features results in an unexpectedly advantageous combination of strong anchoring of the prosthesis in the intervertebral space and easy removal in the event of replacement.

The body of the intervertebral implant according to the invention can be made of a standard material, for example a metal alloy or a ceramic material.

### BRIEF DESCRIPTION OF THE DRAWINGS

The invention is described below on the basis of an advantageous illustrative embodiment and with reference to the attached drawings, in which:

FIG. 1 shows a view of an intervertebral implant according to the invention from above;

FIG. 2 shows a section along the line A-A in FIG. 1;

FIG. 3 shows a section along the line B-B in FIG. 1;

FIG. 4 shows an enlarged detail from FIG. 3; and

FIG. 5 shows the view from FIG. 4 together with specified bone tissue.

### DETAILED DESCRIPTION OF THE INVENTION

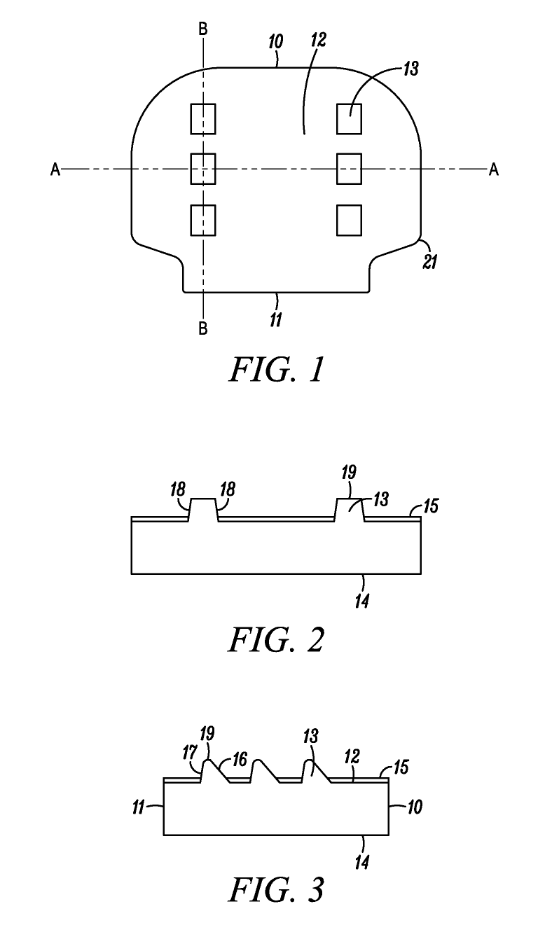

An intervertebral implant shown in FIG. 1 is designed to be inserted with its dorsal end 10 leading into an intervertebral space. During insertion, the intervertebral implant is maneuvered by an instrument that engages on the ventral end 11 of the intervertebral implant. When the implant is inserted into the intervertebral space, a contact face 12 bears on a vertebral body surface that defines the intervertebral space. The implant according to the invention can cooperate with further prosthesis components, for example in order to form an intervertebral prosthesis that simulates the function of an intervertebral disk. For this purpose, the underside 14 of the implant, oriented away from the contact face 12, can be designed as a slide surface that forms a hinge with a slide surface of the other prosthesis component.

Six teeth 13 are formed on the contact face 12 and protrude from the contact face 12. The teeth 13 are arranged in two rows that each comprise three teeth 13 and are oriented substantially in the AP direction. When the implant is inserted into the intervertebral space, the teeth 13 penetrate into the bone tissue of the adjacent vertebral body, such that the contact face bears on the surface of the vertebral body. The contact face 12 is covered by a layer 15 with which the bone tissue can form an intimate connection. The teeth 13 stand free from the layer 15. Apart from the teeth 13, the contact face 12 extends across the entire surface area enclosed by the contour 21 of the implant.

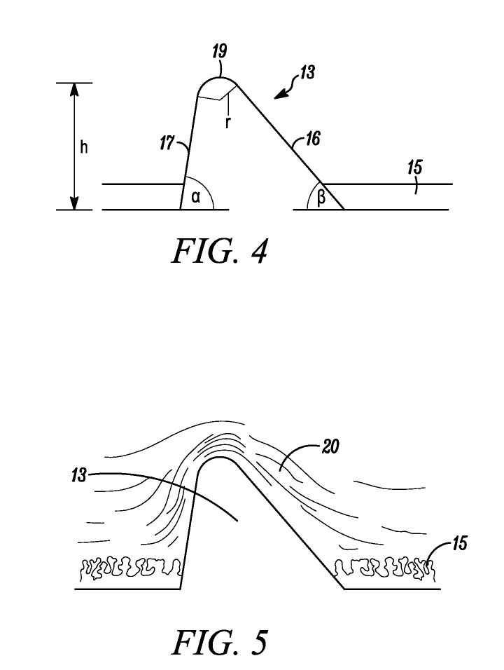

Each tooth 13 has a dorsal flank 16, a ventral flank 17, and two lateral flanks 18. The dorsal flank 16 merges into the ventral flank 17 via a rounded edge 19. The edge 19 extends across the width of the tooth 13 from one lateral flank 18 to the other lateral flank 18. The ventral flank 17 of the tooth 13 has a pitch, indicated by the angle α in FIG. 4, of 82° relative to the horizontal plane of the implant. The pitch of the dorsal flank 16, indicated by the angle β, is approximately 40°. The lateral flanks 18 have a pitch of 82° relative to the horizontal plane, exactly like the ventral flank 17. The teeth 13 have a height h of 1.1 mm above the contact face 12. The teeth protrude by 0.9 mm from the 0.2 mm thick coatings 15. The rounding of the edge 19 has a radius r of 0.1 mm.

As is indicated in FIG. 5, the coating 15 is so porous that the bone tissue 20, likewise indicated in FIG. 5, of the vertebral body can form an intimate connection with the coating 15. The bone tissue 20 is not cut through by the penetrating tooth 13 but merely compressed.

1-11. (canceled)

12. An intervertebral implant for insertion into an intervertebral space comprising:
a contact face for connection to an adjacent vertebral body that bears on bone tissue of an endplate of the adjacent vertebral body;
a porous coating applied to the contact face that allows the bone tissue to grow into pores of the porous coating to form an intimate connection between the contact face and the bone tissue; and
teeth that protrude from the contact face and the porous coating, the teeth having a tip with a rounded edge having a radius of not less than 0.1 mm, the teeth occupying less than 15% of a surface area of the contact face, wherein the rounded edge is configured to compress the bone tissue but not to penetrate through the bone tissue.

13. The implant of claim 12, wherein the teeth occupy less than 10%, preferably less than 8% of the surface area of the contact face.

14. The implant of claim 12, wherein the teeth are configured with a dorsal flank, a ventral flank, and lateral flanks wherein the dorsal flank has a pitch β relative to the contact face which is less than a pitch α of the ventral flank, and the lateral flanks have a pitch relative to the contact face of less than 90°.

15. The implant of claim 14, wherein the pitch β of the dorsal flank is from 40° to 60°, preferably about 40°.

16. The implant of claim 14, wherein the pitch α of the ventral flank is from 78° to 88°, preferably about 82°.

17. The implant of claim 14, wherein the lateral flanks and the ventral flank have the same pitch.

18. The implant of claim 14, wherein the dorsal flank, ventral flank, and lateral flanks have a roughness of less than 12 μm, preferably less than 8 μm.

19. The implant of claim 12, wherein the teeth have a height above the contact face from 1.5 mm to 0.8 mm.

20. The implant of claim 12, wherein the teeth have a width of less than 3 mm, preferably less than 2 mm.

21. The implant of claim 12, wherein the porous coating is applied with a thickness of 0.2 mm.

22. The implant of claim 12, wherein the porous coating is provided with a pore width from 30 μm to 70 μm, and with a pore coverage from 20% to 40% of the contact face.

23. The implant of claim 12, wherein the teeth are arranged in at least two rows that are oriented in an anterior to posterior direction of implantation

24. The implant of claim 12, wherein the contact face is curved to correspond to the shape of the adjacent vertebral body when viewed in an anterior to posterior direction of implantation.

25. The implant of claim 12, wherein the implant is made of at least one of a metal alloy or a ceramic material.

26. The implant of claim 12, wherein the implant is formed with a slide surface on an underside of the implant that cooperates with a further implant to form a hinge surface to simulate the function of an intervertebral disc.

27. A method for securing an intervertebral implant in an intervertebral space comprising:
gaining access to the intervertebral space;
selecting an implant comprising a contact face for connection to an adjacent vertebral body that bears on bone tissue of an endplate of the adjacent vertebral body, a porous coating applied to the contact face that allows the bone tissue to grow into pores of the porous coating to form an intimate connection between the contact face and the bone tissue, and teeth that protrude from the contact face and the porous coating, the teeth having a tip with a rounded edge having a radius of not less than 0.1 mm, the teeth occupying less than 15% of a surface area of the contact face; and
inserting the implant such that the contact face bears on the bone tissue to form an intimate connection, and the teeth bear on the bone tissue to compress the bone tissue but not to penetrate through the bone tissue of the endplate of the adjacent vertebral body.

28. The method of claim 27, further comprising
removing the implant at a later time by breaking the intimate connection between the contact face and the bone tissue.

29. The method of claim 28, further comprising
inserting a replacement intervertebral implant in the intervertebral space.
